(19) United States
(12) Patent Application Publication
(10) Pub. No.: US 2023/0321025 A1
(43) Pub. Date: Oct. 12, 2023

(54) METHOD AND COMPOUND FOR THE TREATMENT AND ELIMINATION OF AN IMMUNODEFICIENCY CONDITION

(71) Applicants: Lydia SEIFU, Silver Spring, MD (US); Seifu BELAY, Greenbelt, MD (US)

(72) Inventors: Lydia SEIFU, Silver Spring, MD (US); Seifu BELAY, Greenbelt, MD (US)

(21) Appl. No.: 18/012,908

(22) PCT Filed: Jun. 23, 2021
(86) PCT No.: PCT/US2021/038772

§ 371 Date: Dec. 23, 2022

Related U.S. Application Data
(60) Provisional application No. 63/042,736, filed on Jun. 23, 2020.

(51) Int. Cl. A61P 31/18; A61K 31/235

(52) U.S. Cl. CPC A61K 31/235; A61P 31/18

(57) ABSTRACT

A method for treatment by boosting an immune system of a subject infected with an immunodeficiency disease, including administering to the subject in need thereof of an anti-pathogenic compound, such that the anti-pathogenic compound uses multiple modes of action against the immunodeficiency disease.

### CROSS REFERENCE TO RELATED APPLICATION

This application claims the benefit of, and incorporates by reference, U.S. Provisional Pat. 63/042,736, entitled “Method and Compound for the Treatment and Elimination of an Immunodeficiency Condition,” which was filed on Jun. 23, 2020.

### BACKGROUND

### 1. Field

The present general inventive concept relates generally to treatment of a viral disease, and particularly, to a method and compound for the treatment and elimination of an immunodeficiency condition.

### 2. Description of the Related Art

AIDS has been reported to be first identified in 1981 among homosexual men and intravenous drug users in New York and California. Shortly after its detection in the United States, evidence of AIDS epidemics grew among heterosexual men, women, and children in sub-Saharan Africa. AIDS quickly developed into a worldwide epidemic, affecting virtually every nation. It is reported that by 2000, an estimated 34.7 million adults and 1.4 million children worldwide, had HIV infection or AIDS. The World Health Organization (WHO), a specialized agency of the United Nations (UN), estimates that from 1981 to the end of 2000 about 21.8 million people died as a result of AIDS. More than 4.3 million of those who died were children under the age of 15.

In the United States, about 40,000 new HIV infections occur each year. More than thirty percent of the infections occur in women, and sixty percent occur in ethnic minorities. About 800,000 to 900,000 U.S. residents were infected with HIV, and about 300,000 people were living with full-blown AIDS, in 2000. In Canada, about 4,200 new HIV infections occur each year. About twenty-three percent of these infections occur in women. About 40,000 Canadians were living with HIV infection or full-blown AIDS in 2000.

The incidence of new cases of HIV infections and AIDS deaths has significantly decreased in Canada and the United States since 1995. This decrease is attributed to the availability of new drug treatments and public health programs that target people most at risk for infection. But while the overall rate of HIV infection seems to be on a downturn, certain populations appear to be at greater risk for the disease. In the United States in 1987, Caucasians accounted for sixty percent of AIDS cases, and blacks and Hispanics only thirty-nine percent. But by 2000 the trend had reversed: thirty-eight percent of new cases were diagnosed in Caucasians and sixty-one percent in blacks and Hispanics. Likewise the number of female AIDS patients in the United States has increased significantly in recent years, from seven percent of all AIDS cases in 1985 to thirty percent in 2000. In the United States, African American and Hispanic women accounted for eighty-two percent of AIDS cases among women in 2000.

In western Europe the first cases of AIDS were detected in the early 1980s, and by the late 1990s, at least 30,000 new HIV infections occurred each year. In 2000, more than 540,000 western Europeans were HIV positive, and twenty percent of these cases were women. Before the dissolution of the Union of Soviet Socialist Republics (USSR) in 1991, eastern Europe reported few HIV cases. But since 1995, HIV infection has spread rapidly in cities of several eastern European countries, including Ukraine, Belarus, and Moldova. The WHO estimates that the total number of HIV infections in this region may have risen from less than 30,000 in 1995 to more than 700,000 in 2000.

While cases of AIDS have been reported in every nation of the world, the disease affects some countries more than others. More than ninety-five percent of all HIV-infected people live in the developing world. In these areas, the disease has sapped the populations of young men and women who form the foundation of the labor force. Most die while in the peak of their reproductive years. Moreover, the epidemic has overwhelmed health-care systems, increased the number of orphans, and caused life expectancy rates to plummet. These problems have reached crisis proportions in some parts of the world already burdened by war, political upheaval, or unrelenting poverty.

Nowhere is this better demonstrated than in sub-Saharan Africa, where the number of AIDS cases far exceeds that of all other geographic regions. Of the estimated 16,000 HIV infections that occur each day worldwide, 7,500 of them occur in sub-Saharan Africa. More than seventy percent of all people infected with HIV live in this region. In some countries in the southern part of the continent, including Botswana, Namibia, Swaziland, and Zimbabwe, as much as twenty-five percent of the population has HIV infection or AIDS.

In Asia, the rates of HIV infection remain low relative to many other areas, but the number of reported cases markedly increased in recent years. Health officials fear the virus will affect more people if it spreads through China and India, the world’s two most populous countries. For example, 1992 marked the first reported cases of HIV infection in India. By the end of 1999 nearly 4 million adults in India were HIV positive. These cases were mostly confined to 10 of the nation’s states, while the remaining 24 states reported low infection rates. HIV infection in India initially was reported primarily in sex workers, but it has quickly spread to the general population in less than five years. Health officials fear that without public education programs, cases of HIV infection will escalate over the next decade, causing the AIDS epidemic in India to dwarf the problems seen in sub-Saharan Africa.

In 2002, the Chinese government reported that China had 850,000 HIV-positive people in a population of more than 1 billion. However, public health experts are concerned by the fast-rising number of new infections among intravenous drug users who share infected needles. In 2000, HIV prevalence among intravenous drug users ranged from forty-four percent to eighty-five percent in selected communities of drug users in both Yunnan, in southern China, and Xinjiang, in northwestern China. The incidence of HIV infection will likely be exacerbated by the growing sex industry in China. Surveys indicate that there are as many as 4 million sex workers in China. Of these, five out of ten never use a condom to protect themselves or their clients from HIV infection or other sexually transmitted infections. In rural areas of China, the incidence of HIV infection is rising because many poverty-stricken people regularly sell their blood. The people who buy the blood use unsterile methods to draw blood, including reusing contaminated needles, which can spread HIV infection.

In Latin America and the Caribbean, region nearly 1.8 million people have been diagnosed with HIV infection or AIDS, twice the incidence in the United States and Canada. In Mexico, 150,000 people have been diagnosed with HIV infection or AIDS, and the disease is the third leading cause of death in men aged 20 to 34. Honduras, which accounts for less than a fifth of the population in Central America, reports more than half of the AIDS cases in that region. In the state of Sao Paolo, Brazil, AIDS has been the leading cause of death among women aged 20 to 34 since 1992.

The following includes information from “The Extended Impact of Human Immunodeficiency Virus/AIDS Research.” (See https://doi.org/10.1093/infdis/jiy441).

Human immunodeficiency virus (HIV) is one of the most extensively studied viruses in history. While there have been substantial investments in HIV/AIDS research conducted, since over 37 years ago when first reported in the United States, this virus stands as one of the most complicated viruses to date in 2020. HIV-1 is still considered by the World Health Organization (WHO) a pandemic, crippling lives, economies, and in many developing nations a large contributing factor to the continued breakdown in infrastructure.

The first cases of AIDS were reported in the United States 37 years ago. Since then, over 77 million people have been infected worldwide, resulting in over 35 million deaths. Currently, there are 36.9 million people living with human immunodeficiency virus (HIV), 1.8 million new infections, and nearly 1 million AIDS-related deaths annually. Billions of research dollars have been invested toward understanding, treating, and preventing HIV infection. The largest funder of HIV/AIDS research is the National Institutes of Health (NIH), investing nearly $69 billion in AIDS research from fiscal years 1982-2018. Despite the staggering disease burden, the scientific advances directly resulting from investments in AIDS research have been extraordinary. HIV is one of the most intensively studied viruses in history, leading to an in-depth understanding of viral biology and pathogenesis. However, despite the investments in further understanding the virus, and development of drugs such as azidothymidine (AZT), treatment has been proven to only partially and temporarily suppress virus replication.

HIV is a virus that attacks the immune system. Specifically, HIV targets CD4 white blood cells, which is the body’s principal defenders against infection, using them to make copies of themselves.

The following is an excerpt from “THE SCIENCE OF HIV AND AIDS -OVERVIEW.” (See https://www.avert.org/professionals/hiv-science/overview).

### The Life-Cycle of HIV:

FIG. 1 illustrates a life cycle of HIV.

### 1. Attachment and Entry

The process of producing new viruses begins when HIV gains entry to a cell. This process happens in two stages, attachment and fusion. When HIV makes contact with a CD4 cell, the gp120 spikes on the surface of HIV lock onto the CD4 receptor and another co-receptor, either CCR5 or CXCR4. The gp41 protein is used to fuse the HIV envelope with the cell wall. This process of fusion allows the HIV capsid to enter the CD4 cell.

Several types of antiretroviral drug have been developed to block different stages of the processes of attachment and entry: CCR5 inhibitor, Attachment inhibitor, and Fusion inhibitor.

### 2. Reverse Transcription

When HIV RNA enters the cell it must be ‘reverse transcribed’ into proviral DNA before it can be integrated into the DNA of the host cell. HIV uses its reverse transcriptase enzyme to convert RNA into proviral DNA inside the cell. Two types of antiretroviral drug have been developed to stop the action of reverse transcriptase and the creation of proviral DNA: Nucleoside and nucleotide reverse transcriptase inhibitors (NRTIs and NtRTIs) block HIV production by inserting a nucleoside or nucleotide into the chain of HIV DNA as it is created, terminating the chain.

### 3. Integration

After HIV RNA is converted into DNA, HIV’s integrase enzyme attaches itself to the end of the proviral DNA strands and it is passed through the wall of the cell nucleus. Once the proviral DNA enters the cell nucleus, it binds to the host DNA and then the HIV DNA strand is inserted into the host cell DNA.

### 4. Transcription and Translation

The cell will produce HIV RNA if it receives a signal to become active. CD4 cells become activated if they encounter an infectious agent. When the cell becomes active, HIV uses the host enzyme RNA polymerase to make messenger RNA. This messenger RNA provides the instructions for making new viral proteins in long chains. The long chains of HIV proteins are cut into smaller chains by HIV’s protease enzyme.

### 5. Assembly and Budding

These protein chains begin to assemble into new viruses at the cell wall. HIV protease inhibitors are designed to block the activity of HIV’s protease enzyme. As the virus buds from the cell wall, its genome becomes enclosed in a capsid produced from HIV’s gag protein. After the new virus is assembled, it must leave the cell by pushing through the cell wall. To leave the cell completely and become infectious, the virus must take lipids (fats) from the cell wall to make the surface glycoproteins. Maturation inhibitors are being developed to block the cutting of the gag protein that is needed to produce a mature virus.

HIV as we know is a spherical retrovirus, with an outer shell called the envelope covered in spikes of “glycoproteins” gp120 and gp41, allowing HIV to lock onto the CD4 receptor on CD4 T cells. The nucleus of the virus, is held in the capsid containing two enzymes essential for HIV replication, the reverse transcriptase and integrase molecules, and two strands of RNA holding HIV’s genetic material.

HIV’s RNA is made up of nine genes which contain all the instructions to make new viruses. Three of these genes ― gag, poland env - provide the instructions to make proteins that will form new virus particles. For example, env provides the code to make the proteins that form the envelope, or shell, of HIV. gag makes the structural proteins such as the matrix and the capsid, and pol makes the enzymes that are essential for making new viruses.

The other six genes, known as tat, rev, nef, vif, vpr and vpu, provide code to make proteins that control the ability of HIV to infect a cell, produce new copies of virus or release viruses from infected cells.

HIV-1 Groups and subtypes; Group M, N, O and P, 6 possible Reading Frame (RFs) of HIV complete genome. Sub types of M Clades: A (West Africa), B (America & Europe), C (S&E Africa) dominate Clade in Ethiopia, D (E&C Africa), E (Recombinant), F (Cen. Africa & S America), G (Africa and Ce. Europe), H (Cen. Africa, J (Africa, Caribbean), K Democratic Republic of Congo).

Historically, the recognized treatment for HIV-1 infection is nucleoside analogs, inhibitors of HIV-1 reverse transcriptase (RT). Intervention with these antiretroviral agents has led to a decline in the number of reported AIDS cases and has been shown to decrease morbidity and mortality associated with advanced AIDS. Prolonged treatment with these reverse transcriptase inhibitors eventually leads to the emergence of viral strains resistant to their antiviral effects. Recently, inhibitors of HIV-1 protease have emerged as a new class of HIV-1 chemotherapy. HIV-1 protease is an essential enzyme for viral infectivity and replication. Protease inhibitors have exhibited greater potency against HIV-1 in vitro than nucleoside analogs targeting HIV-1 RT. Inhibition of HIV-1 protease disrupts the creation of mature, infectious virus particles from chronically infected cells. This enzyme has become a viable target for therapeutic intervention and a candidate for combination therapy.

Knowledge of the structure of the HIV-1 protease has led to the development of novel inhibitors such as saquinovir, ritonavir, indinivir and nelfinavir. NNRTIs (non-nucleoside reverse transcriptase inhibitors) have recently gained an increasingly important role in the therapy of HIV infection. Several NNRTIs have proceeded onto clinical development (ie, tivirapine, loviride, MKC-422, HBY-097, DMP 266). Nevirapine and delaviridine are already licensed for clinical use. Every step in the life cycle of HIV-1 replication is a potential target for drug development.

Unfortunately, however, the single targeted approach as stated above, has only been able to target RT or PR enzymes. As a result, this approach has favored the virus natural selection to evade drug therapy by mutating at a single or few amino acid sequences. According to a CDC report (December 2001), 75% mortality rate in HIV-1 patients is co-related to drug resistant HIV variants. The search for new antiretroviral effective drug against HIV-1 resistant variants has dramatically increased.

Many of the antiretroviral drugs currently used in chemotherapy either are derived directly from natural products, or are synthetics based on a natural product model. The rationale behind the inclusion of deoxynucleoside as a natural based antiviral drugs originated in a series of publications dating back as early as 1950, wherein the discovery and isolation of thymine pentofuranoside from the air-dried sponges (cryptotethia crypta) of the Bahamas was reported. A significant number of nucleosides were made with regular bases but modified sugars, or both acyclic and cyclic derivatives, including AZT and acyclovir. The natural spongy-derived product led to the first generation, and subsequent second-third generations of nucleosides (AZT, DDI, DDC, D4T, 3TC) antivirals specific inhibitors of HIV-1 RT.

A number of non-nucleoside agents (NNRTIs) have been discovered from natural products that inhibit RT allosterically. NNRTIs have considerable structural diversity but share certain common characteristics in their inhibitory profiles. Among NNRTIs isolated from natural products include: calanoid A from calophylum langirum; Triterpines from Maporonea African a. There are publications on natural HIV integrase inhibitors from the marine ascidian alkaloids, the lamellarin.

The role of HIV protease in the production of functionally infectious particle has been described as a critical process for retrovirus as well as HIV replication. The natural product, Pepstatin A, is a metabolite of streptomycin testaceus and Streptomyces argentolus var. toyonakensis was shown to inhibit HIV-1 Protease enzyme. The key strategy used in the development of the current HIV-1 protease inhibitors was to substitute the scissile P1-P1 amide bond by a nonhydrozable isoster with tetrahedral geometry; the designs were guided by assays and based on substrate specificity. It eventually led to the optimization of peptidomimetric lead structure and the development of novel class of protease inhibitors including indinvir, Saqunovir, nelfinavir and retinovir.

In Ethiopia, there are currently more than 3 million adults and close to 1 million children infected with HIV-1. The rate of HIV-1 vertical and horizontal transmission has drastically increased over the years. More than 50% of the nation’s available hospital beds are over crowded with HIV-1 patients, and 99.9% of the HIV-1 patients cannot afford the commercially available antiretroviral drugs. Even for those who can afford it (<0.1%) there is no HIV-1 staging or managing infrastructure to evaluate therapeutic indices. The national health status is in a state of emergency that could cripple the national economy and decimate the younger generations. Unless immediate therapeutic and behavioral interventions are expedited, the exponential rate of HIV-1 growth and related morbidity is easily over six million. This is a deadly reality that Ethiopians, Ethiopian HIV-1 experts, and the world at large are currently confronted with. It is within this background of multiple challenges that Bio-TSquare LLC presents this alternative proposal. The rational for Bio-T Square to undertake this project is to seek a collective solution with Ethiopian Government, Ethiopian and International HIV-1 experts. Seeking an alternative solution based on science and collaborative work is the core of this proposition. The following summary explains the problems with drug therapy in Ethiopia and the significance of our undertaking to the Ethiopian AIDS Patients and to the Ethiopian economy.

In order to provide therapeutic intervention to 400,000 AIDS infected patients with the commercially available drugs, the country of Ethiopia alone will have to spend billions of dollars per year. This figure does not include other concomitant drugs needed for TB, abdominal fungus, pneumonia, toxic effects caused by drugs and monthly patient evaluation for therapeutic index and HIV-1 dose response assessments. This approach is simply way beyond the GNP of Ethiopia.

The Sentimental National HIV-1 Epidemics Survey (2001) shows the severity of HIV-1 epidemics in Ethiopia. Also, The HIV-1 symposium held at the UN conference center (2003) has also shown the depth and the magnitude of HIV-1 catastrophe in the youth generation and the armed forces. These presentations have a questionably shown that HIV-1 epidemics have threatened the survival of the nation in many aspects. The fact that HIV-1 viral mayhem took the lives of 150,000 in one year alone show catastrophic emergency signal unless aggressive therapeutic intervention is conducted.

The other serious problem arising from importing and prescribing non-HART (highly active retrovirus therapy) drug treatments in Ethiopia is the creation of fertile environment for the emergence of highly virulent, resistant viruses. More than 75% of AIDS therapeutic failure is caused by resistance viruses. A centralized data bank and HIV-1 management team is crucial to monitor the success or failure of antiretroviral therapy. Salvage therapy with the commercial drugs, triple or quadruple combination is essential to reach effective clinical diagnostic therapeutic index. This again could bankrupt the economy of Ethiopia.

In view of the drawbacks associated with conventional remedies and the growing worldwide concern for the AIDS epidemic, there is a need for a remedy which is inexpensive, less toxic, potent, and easily available. In this regard, the inventors of the present invention believe that the compound or antiviral agent of the invention, H2K1001(90I or 90i), would be a perfect alternative since it is less costly, highly potent, easy to deliver to AIDS patients, and highly active against resistant viruses. In addition, the compound of the invention is a simple multi-charged molecule that could be manufactured at low cost. Consequently, the cost of the drug would be affordable to the majority of AIDS patients in Ethiopia and other developing countries.

Therefore, there is a need for an effective remedy and/or drug that is natural, inexpensive, and non-toxic. As such, there is a need for a method and compound for the treatment and elimination of an immunodeficiency condition.

### SUMMARY

The principal object of the present invention is to provide a method and compound for the prophylaxis or treatment of an immunodeficiency condition, such as acquired immunodeficiency syndrome (AIDS).

An object of the present invention is to provide a method and compound for the prophylaxis or treatment of an immunodeficiency condition, such as acquired immunodeficiency syndrome (AIDS), wherein the compound is less toxic and more potent at lower doses (or lower concentrations) than the conventional compounds.

Another object of the present invention is to provide a method and compound for the prophylaxis or treatment of an immunodeficiency condition, such as acquired immunodeficiency syndrome (AIDS), wherein the compound is effective in advanced, as well as in early stages of the condition or infection.

An additional object of the present invention is to provide a method and compound for controlling or arresting replication of an immunodeficiency virus, such as human immunodeficiency virus (HIV).

Yet an additional object of the present invention is to provide a method and compound for the prophylaxis or treatment of an immunodeficiency condition, such as acquired immunodeficiency syndrome (AIDS), wherein the compound comprises an herbal extract. The extract includes a glycol derivative. More specifically, the glycol derivative includes diethylene glycol dibenzoate.

Still yet an additional object of the present invention is to provide a method and compound for the prophylaxis or treatment of an immunodeficiency condition, such as acquired immunodeficiency syndrome (AIDS), which includes a glycol derivative. More specifically, the glycol derivative includes diethylene glycol dibenzoate.

A further object of the present invention is to provide a method and compound for the prophylaxis or treatment of an immunodeficiency condition, such as acquired immunodeficiency syndrome (AIDS), which are inexpensive, highly potent, easy to administer to the patients, highly active against the resistant virus, and easy to manufacture.

In accordance with the present invention, a compound for the prophylaxis or treatment of an infection or condition caused by an immunodeficiency virus, includes a glycol derivative. More specifically, the glycol derivative includes diethylene glycol dibenzoate.

In accordance with the present invention, a method for the prophylaxis or treatment of an infection or condition caused by an immunodeficiency virus, includes administering to a subject in need thereof a compound including a glycol derivative. More specifically, the glycol derivative includes diethylene glycol dibenzoate.

In summary, the compound of the invention may be known as H2K1001(90I or 90i), but is not limited thereto, and is a highly potent new antiretroviral drug or agent that works against resistant HIV viruses. Initial evaluation of this compound in the CEMSS cell line demonstrated significant antiviral activity against the RF laboratory strain of HIV-1. The purified fraction 90I (or 90i) yielded a 50% effective concentration (EC50) of 0.02 ug/ml. At the same time, there was no discernible drug induced toxicity in this study (50% inhibitory concentration, IC50, was greater than the high test concentration of 100 ug/ml). The resulting therapeutic index (TI=IC50/EC50) of greater than 5000 suggests a highly active compound. Testing in PBMC against primary clinical isolates of HIV-1 P2, which is resistant to a variety of non-nucleoside inhibitors or HIV-1 reverse transcriptase. Also, the inventive compound (H2K1001(90I or 90i)) produced an EC50 of 0.8 ug/ml, an IC50 greater than 100, and a Tl of greater than 125.

The present general inventive concept provides a method and compound for the treatment and elimination of an immunodeficiency condition.

Additional features and utilities of the present general inventive concept will be set forth in part in the description which follows and, in part, will be obvious from the description, or may be learned by practice of the general inventive concept.

The foregoing and/or other features and utilities of the present general inventive concept may be achieved by providing a method for controlling replication of an immunodeficiency virus, consisting essentially of exposing a cell infected with an immunodeficiency virus to an antiviral agent, and wherein the antiviral agent is diethylene glycol dibenzoate.

The cell may comprise a lymphocyte.

The immunodeficiency virus may comprise a human immunodeficiency virus (HIV).

The cell may comprise a CD4+T Cell.

The foregoing and/or other features and utilities of the present general inventive concept may also be achieved by providing a method for the treatment of an immunodeficiency condition, consisting essentially of administering to a subject in need thereof an antiviral agent, and wherein the antiviral agent is diethylene glycol dibenzoate.

The immunodeficiency condition may comprise acquired immunodeficiency syndrome (AIDS).

The foregoing and/or other features and utilities of the present general inventive concept may also be achieved by providing a method for the treatment of an infection caused by an immunodeficiency virus, consisting essentially of administering to a subject in need thereof an antiviral agent, and wherein the antiviral agent is diethylene glycol dibenzoate.

The immunodeficiency virus may comprise a human immunodeficiency virus (HIV).

The infection may comprise acquired immunodeficiency virus syndrome (AIDS).

The foregoing and/or other features and utilities of the present general inventive concept may also be achieved by providing a method for treatment by boosting an immune system of a subject infected with an immunodeficiency disease, including administering to the subject in need thereof of an anti-pathogenic compound, such that the anti-pathogenic compound uses multiple modes of action against the immunodeficiency disease.

The anti-pathogenic compound may be diethylene glycol dibenzoate.

The anti-pathogenic compound may use the multiple modes of action against the immunodeficiency disease is at least one of inhibition of reverse transcriptase and protease, revitalizing at least one of a dysfunctional monocyte and a dysfunctional macrophage, modulating an immune cell signal switch from Th2 to Th1, and stimulating production of gamma interferon.

The immunodeficiency disease may be caused by HIV.

The anti-pathogenic compound may prevent at least one of early fusion, late, and both early and late stages of HIV.

The foregoing and/or other features and utilities of the present general inventive concept may also be achieved by providing a method of eliminating an immunodeficiency disease from a subject, including administering to the subject in need thereof of an anti-pathogenic compound, such that the anti-pathogenic compound restores operation of an immune system.

The anti-pathogenic compound may be diethylene glycol dibenzoate.

The anti-pathogenic compound may restore operation of the immune system by inhibiting reverse transcriptase and protease.

The anti-pathogenic compound may restore operation of the immune system by boosting the immune system.

The anti-pathogenic compound may boost the immune system by revitalizing at least one of a dysfunctional monocyte and a dysfunctional macrophage.

The anti-pathogenic compound may boost the immune system by stimulating production of gamma interferon.

The anti-pathogenic compound may boost the immune system by modulating an immune cell signal switch from Th2 to Th1.

The immunodeficiency disease may be caused by HIV.

The anti-pathogenic compound may prevent at least one of early fusion, late, and both early and late stages of HIV.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

These and/or other features and utilities of the present generally inventive concept will become apparent and more readily appreciated from the following description of the embodiments, taken in conjunction with the accompanying drawings of which:

FIG. 1 illustrates a life cycle of HIV;

FIG. 2 is a graphical representation illustrating the effect of the compound of the present invention, H2K1001(90I or 90i), on HIV-1 in CEM-SS T-lymphoblastoid cells;

FIG. 3 is a graphical representation illustrating the effect of control compound (AZT) on HIV-1 in CEM-SS T-lymphoblastoid cells;

FIG. 4 is a graphical representation illustrating the effect of the compound of the present invention, H2K1001(90I or 90i), on HIV-1 (P2) in peripheral blood nonnuclear cells (PBMC);

FIG. 5 is a graphical representation illustrating the effect of control compound (AZT) on HIV-1 in PBMC;

FIG. 6A illustrates a molecular structure of an anti-pathogenic compound, known as 90I (diethylene glycol dibenzoate), according to an exemplary embodiment of the present general inventive concept;

FIG. 6B illustrates a molecular structure of the anti-pathogenic compound, known as 90I, according to an exemplary embodiment of the present general inventive concept;

FIG. 7 illustrates a graph showing an effect of 90I compared to AZT on sensitive HIV RF strain in CEM-SS cells, according to an exemplary embodiment of the present general inventive concept;

FIG. 8 illustrates a graph showing inhibitory activity of 90I compared to AZT on an MDR-27221 HIV strain, according to an exemplary embodiment of the present general inventive concept;

FIG. 9 illustrates a graph showing inhibitory activity of 90I compared to AZT on an K103N/P2 resistant HIV strain to NRTI and NNRTI Drugs in PBMC, according to an exemplary embodiment of the present general inventive concept;

FIG. 10 illustrates a graph showing inhibitory activity of 90I compared to AZT on an monotropic/neurotropic HIV strain, according to an exemplary embodiment of the present general inventive concept;

FIG. 11 illustrates a graph showing antiretroviral mode of action of 90I compared to AZT and Indinavir, according to an exemplary embodiment of the present general inventive concept;

FIG. 12 illustrates a graph showing a combination of drug therapy using 90I and AZT, according to an exemplary embodiment of the present general inventive concept;

FIG. 13 illustrates a graph showing 90I effect on chronically infected H9 cells, according to an exemplary embodiment of the present general inventive concept; and

FIG. 14 illustrates a graph showing production of gamma interferon by cells receiving 90I compared to other drugs, according to an exemplary embodiment of the present general inventive concept.

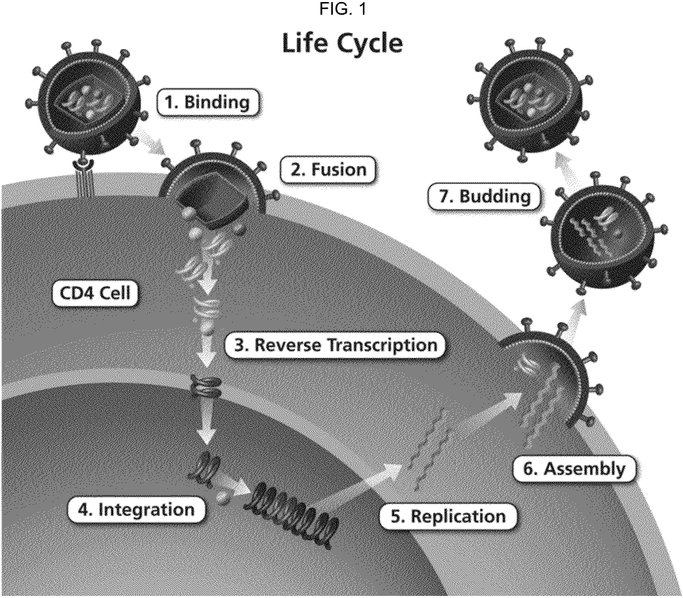

### DETAILED DESCRIPTION

Various example embodiments (a.k.a., exemplary embodiments) will now be described more fully with reference to the accompanying drawings in which some example embodiments are illustrated. In the figures, the thicknesses of lines, layers and/or regions may be exaggerated for clarity.

Accordingly, while example embodiments are capable of various modifications and alternative forms, embodiments thereof are shown by way of example in the figures and will herein be described in detail. It should be understood, however, that there is no intent to limit example embodiments to the particular forms disclosed, but on the contrary, example embodiments are to cover all modifications, equivalents, and alternatives falling within the scope of the disclosure. Like numbers refer to like/similar elements throughout the detailed description.

It is understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art. However, should the present disclosure give a specific meaning to a term deviating from a meaning commonly understood by one of ordinary skill, this meaning is to be taken into account in the specific context this definition is given herein.

The present invention is based on the discovery that an herbal extract, which includes a glycol derivative, is effective against HIV virus. More specifically, we found that the extract includes diethylene glycol dibenzoate, which in vitro testing showed to be effective against HIV-1 virus.

We examined the antiviral activity of an antiviral agent or compound that we have designated as 90I (or 90i), although this name is not limited thereto, and which was derived from an extract of an herbal mixture that we designated as H2K1001, against the RF laboratory strain of HIV-1 in CEM-SS T-lymphoblastoid cells, and against a nucleoside and non-nucleoside resistant clinical isolate of HIV-1 (P2) in peripherial blood mononuclear cells (PBMC). A brief description of each assay employed is discussed below.

H2K1001(90I or 90i) is a natural product that was isolated by bioassay guided fractionations, and further purified, molecularly characterized and tested against variety of HIV-1 strains.

### Antiviral Drug Assay in CEM-SS Cells

This assay was used to test the effectiveness of anti-HIV drug monotherapy in the established T-cell line CEM-SS infected with HIV-1_{RF}, HIV-1_{IIIb}, HIV-2_{ROD}, or other cytolytic variants of HIV. The antiviral agent and control (known antiviral drug–AZT) efficacy and cytotoxicity were determined by the metabolic reduction of the tetrazolium salt MTS (available from Promega).

### Drug and Cell Preparation

The drugs were solubilized in DMSO (dimethyl sulfoxide). Three to ten vials of the antiviral agent and AZT were prepared and maintained at -70° C. CEM-SS cells were in logarithmic growth phase at the time of experimentation. Virus (HIV-1_{RF}) stocks that past the necessary quality controls (killing effect on the type of cells used, limited variability, etc) were thawed and maintained on ice until required for addition.

### Test Plates

Half-log serial dilutions of the antiviral agent or compound of the invention, H2K1001 (90i), were made in tissue culture media (RPMI 1640 without phenol red, supplemented with 10% heat inactivated fetal bovine serum, 1% L-Glutamine, 1% Pen/Strep and 50 ug/ml gentimycin). Virus, cell, drug and diluent (ethanol) controls were included for these tests. Virus was added after cells and drugs were mixed. Cell, drug, and virus containing cultures were incubated for six (6) days without additional feeding at which time the cultures were evaluated macroscopically and microscopically for cell toxicity, and the tetrazolium salt MTS was added for quantitation by the colorimetric determination of formazan production (optical density).

### Endpoint Determinations

Drug efficacy and cytotoxicity effects were determined six days after infection by the metabolic reduction of the tetrazolium salt XTT to its‘ formazan by surviving cells and was quantitated by determination of optical density at 450 nm with background subtraction at 650 nm (Tables 1-4).

### Results

The drug plate analysis reports from this assay are provided below in Tables 1-4. Calculated endpoints (EC50, etc.) were determined using Cho and Cho Median Effect Equation (Daids Virology Manual for HIV Laboratories, Compiled by The Division of Aids, National Institute of Allergy and Infectious Diseases, National Institutes of Health and Collaborating Investigators, Version January 1997).

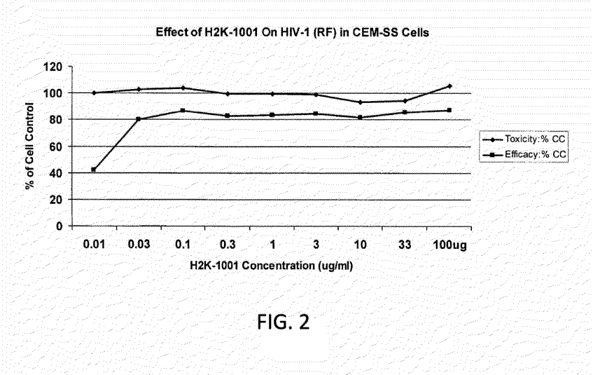

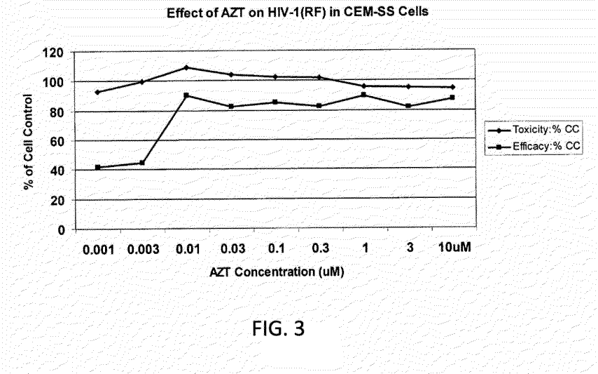

FIGS. 2-3 are graphical representations illustrating the effects of the compound of the present invention and the control drug (AZT), respectively, on HIV-1 in CEM-SS T-lymphoblastoid cells. Tables 5-6 provide the data plotted in FIGS. 2-3, respectively.

Initial evaluation of 90i, the antiviral agent of the invention, in the CEM-SS cell line, demonstrated significant antiviral activity against the RF laboratory strain of HIV-1. The antiviral agent yielded a 50% effective concentration (EC₅₀) of approximately 0.020 ug/ml (see Table 1). At the same time, there was no discernible drug induced toxicity in this study (50% inhibitory concentration, IC₅₀, was greater than the high test concentration of 100 ug/ml) (see Table 2). The resulting therapeutic index (TI=IC₅₀/EC₅₀) of greater than 5000 suggests a highly active compound, at least in vitro. The AZT control in our study (included to ensure the functionality of all antiviral testing) yielded an EC₅₀ of approximately 0.0035 uM (see Table 3), an IC₅₀ greater than 10 uM and a TI of greater than 2857 (see Table 4).

TABLE 1

Efficacy Testing Data for R2K18001 (90I) in CEM-SS T-Lymphoblastsid Cells Versus HIV-1 (RF)

| | 100+ | 33+ | 10+ | 3+ | +1 | 0.3+ | 0.1+ | 0.03+ | 0.01+ | VC+ |
|---|---|---|---|---|---|---|---|---|---|---|
| Mean +/-SD CV % CC | 2.2450 | 2.1945 | 2.0385 | 2.2499 | 2.2771 | 2.1099 | 2.2196 | 2.0839 | 0.9892 | 0.5222 |
| | 1.9588 | 1.9560 | 2.0038 | 1.9311 | 1.9186 | 1.8158 | 2.0140 | 1.6926 | 1.0238 | 0.5293 |
| | 2.1625 | 2.0909 | 1.8968 | 1.9576 | 1.8914 | 2.1016 | 2.0883 | 2.0343 | 1.0477 | 0.4715 |
| | 2.1221 | 2.0805 | 1.9797 | 2.0459 | 2.0264 | 2.0091 | 2.1073 | 1.9368 | 1.0282 | 0.5077 |
| | 0.3473 | 0.1196 | 0.0739 | 0.1764 | 0.2174 | 0.1675 | 0.1041 | 0.2130 | 0.6294 | 0.0315 |
| | 0.0694 | 0.0575 | 0.0373 | 0.0862 | 0.1073 | 0.0833 | 0.0494 | 0.1100 | 0.0288 | 0.0621 |
| | 88 | 86 | 82 | 84 | 84 | 83 | 87 | 80 | 42 | 21 |

*Drug Concentration in ugied.
SD = Studard Deviation.
(“V = Coellicient of VatU_{WC}I_{II}.
CC = Cell Control.
VC = Virus Control.

TABLE 2

Toxicity Testing Data for H2K1001 (90I) in CEM-SS T-Lymphoblastoid Cells Versus HIV-1 (RF)

| | 100* | 33* | 10* | 3* | 1* | 0.3* | 0.1* | 0.03* | 0.01* | CO* |
|---|---|---|---|---|---|---|---|---|---|---|
| Mean +/-SD CV % CC | 2.3986 | 2.3330 | 1.9415 | 2.0028 | 2.0248 | 1.8679 | 2.3026 | 2.1542 | 2.2494 | 2.0675 |
| | 2.6684 | 2.2031 | 2.5097 | 2.5800 | 2.6325 | 2.6984 | 2.5829 | 2.6984 | 2.5627 | 2.5950 |
| | 2.6143 | 2.3276 | 2.3457 | 2.6192 | 2.5957 | 2.6727 | 2.6592 | 2.6382 | 2.4831 | 2.6080 |
| | 2.5604 | 2.2879 | 0.2924 | 2.4007 | 2.4177 | 2.4130 | 2.5149 | 2.4969 | 2.4317 | 2.4235 |
| | 0.3427 | 0.0735 | 0.2924 | 0.3451 | 0.3407 | 0.4722 | 0.1878 | 0.2983 | 0.1628 | 0.3084 |
| | 0.0537 | 0.0321 | 0.1291 | 0.1438 | 0.1409 | 0.1957 | 0.0747 | 0.1195 | 0.0070 | 0.1272 |
| | 106 | 94 | 93 | 99 | 100 | 100 | 104 | 103 | 100 | 100 |

*Drug Concentration in u phs).

TABLE 3

Efficacy TestingData for AZT in CEM-SS T-Lymphoblastied Cells Versus HIV-1 (RF)

| | 10+ | 3+ | 1+ | 0.3+ | 0.1+ | (0.03+ | 0.01+ | 0.003+ | 0.001+ | VC+ |
|---|---|---|---|---|---|---|---|---|---|---|
| Mean +/-SD CV % CC | 1.8513 | 1.9038 | 1.9616 | 1.9402 | 1.8079 | 1.6606 | 1.8216 | 0.8944 | 0.9015 | 0.4326 |
| | 1.8922 | 1.8368 | 2.0118 | 1.7198 | 2.1031 | 1.7129 | 1.5744 | 0.9499 | 0.8874 | 0.4464 |
| | 2.0748 | 1.7308 | 1.9864 | 1.8287 | 1.7718 | 2.1320 | 2.5998 | 1.1112 | 0.9820 | 0.4401 |
| | 1.9394 | 1.8238 | 1.9866 | 1.8296 | 1.8943 | 1.8352 | 1.9986 | 0.9852 | 0.9236 | 0.4397 |
| | 0.1190 | 0.0872 | 0.0251 | 0.1102 | 0.1818 | 0.2584 | 0.5351 | 0.1126 | 0.0510 | 0.0069 |
| | 0.0614 | 0.0478 | 0.0126 | 0.0802 | 0.0959 | 0.1408 | 0.2677 | 0.1143 | 0.0553 | 0.0157 |
| | 87 | 82 | 90 | 82 | 85 | 83 | 90 | 44 | 42 | 20 |

+Drug Concentation is uM.

TABLE 4

Toxicity Testing Data for AZT in CBM-SS T-lysuphoblatiod Cells Versus HIV-1 (RP)

| | 10+ | 3+ | 1+ | 0.3+ | 0.1+ | 0.03+ | 0.01+ | 0.003+ | 0.001+ | CC+ |
|---|---|---|---|---|---|---|---|---|---|---|
| Mean +/-SD CV % CC | 2.2320 | 2.0787 | 1.8131 | 1.6316 | 1.1783 | 2.5698 | 2.1282 | 2.2086 | 1.6705 | 2.0101 |
| | 2.1655 | 2.3588 | 2.4605 | 2.9433 | 2.6482 | 2.2796 | 2.6386 | 2.3516 | 2.1309 | 2.1129 |
| | 1.9163 | 1.9144 | 2.1094 | 2.2144 | 2.3896 | 2.0840 | 2.4680 | 2.0717 | 2.3829 | 2.5305 |
| | 2.1046 | 2.1136 | 2.1277 | 2.2316 | 2.2688 | 2.3111 | 2.4116 | 2.2106 | 2.0614 | 2.2178 |
| | 0.1664 | 0.2287 | 0.3241 | 0.6572 | 0.4477 | 0.2443 | 0.2598 | 0.1400 | 0.3612 | 0.2756 |
| | 0.0791 | 0.1044 | 0.1523 | 0.2904 | 0.1975 | 0.1057 | 0.1077 | 0.0633 | 0.1752 | 0.1243 |
| | 95 | 95 | 96 | 102 | 102 | 104 | 109 | 100 | 93 | 100 |

+Drug Concentration In uM.

TABLE 5

Effect of H2K-1001 on HIV-1 (RF) in CEM-SS Cells

| H2K-1001 ug/ml | Toxicity % CC | Efficacy % CC |
|---|---|---|
| 0.01 | 100 | 42 |
| 0.03 | 103 | 80 |
| 0.1 | 104 | 87 |
| 0.3 | 100 | 83 |
| 1 | 100 | 84 |
| 3 | 99 | 84 |
| 10 | 93 | 82 |
| 33 | 94 | 86 |
| 100 ug | 106 | 88 |

TABLE 6

Effect of AZT on HIV-1 (RT) in CEM-SS Cells

| AZT Concentration (uM) | Toxicity % CC | Efficacy % CC |
|---|---|---|
| 0.001 | 93 | 42 |
| 0.003 | 100 | 44 |
| 0.01 | 109 | 90 |
| 0.03 | 104 | 83 |
| 0.1 | 102 | 85 |
| 0.3 | 102 | 82 |
| 1 | 96 | 90 |
| 3 | 95 | 82 |
| 10 uM | 95 | 87 |

### Antiviral Drug Assay in PBMC

This assay was used to test the effectiveness of anti-HIV drug monotherapy in peripheral blood mononuclear cells (PBMC) infected with clinical isolates of HIV-1. Drug efficacy was determined by the production of supernatant HIV-1 p24. Drug-induced cytotoxicity was determined by the metabolic reduction of tetrazolium salts (MTS; Promega).

### Drug and Cell Preparation

The antiviral drug or agent of the invention, H2K-1001 (90i), was solubilized. PBMCs, stimulated with PHA (phytohemagglutinin) for three days were employed throughout. Virus stocks (U.S. Clinical isolates) grown in PBMCs only, that past the necessary quality controls (i.e., replicate to high titer in PBMCs and meet the requirements as to genotype and phenotype) were rapidly thawed and maintained on ice until required for infection.

### Test Plates

Half-log serial dilutions of test drugs and controls were prepared in tissue culture media (RPMI 1640 without phenol red, supplemented with 10% heat inactivated fetal bovine serum, 1% L-Glutamine, 1% Pen/Strep and 50 µg/ml gentamicin). Virus and cell controls were included on each test plate. Virus was added after cells and drugs were mixed. Cell, drug and virus containing cultures were incubated for seven (7) days with an interim feeding at day three or four.

### Endpoint Determinations (Sample Harvest, P24 Quantitation and Cell Viability)

Supernatants were sampled at seven days for p24 determination by antigen capture ELISA. Cytotoxicity was measured by the metabolic reduction of the tetrazolium salt according to the manufacturer’s (Promega) recommendations.

### Results

The drug analysis reports from these studies are provided below in Tables 7-10. Efficacy (EC₅₀) and viability (IC₅₀) determinations were made using Cho and Cho Median Effect Equation, as noted above. Efficacy is expressed as the 50% effective concentration (EC₅₀) and drug induced toxicity as the 50% inhibitory concentration (IC₅₀).

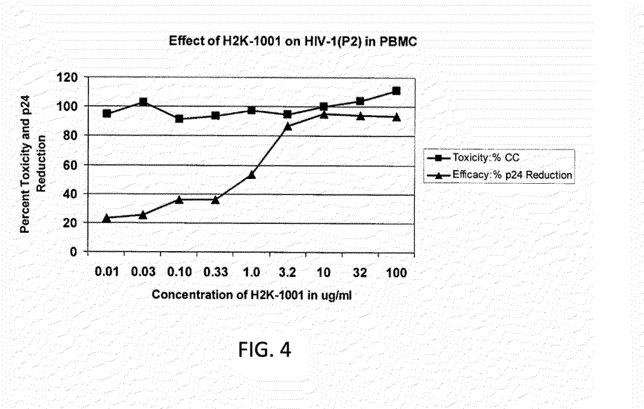

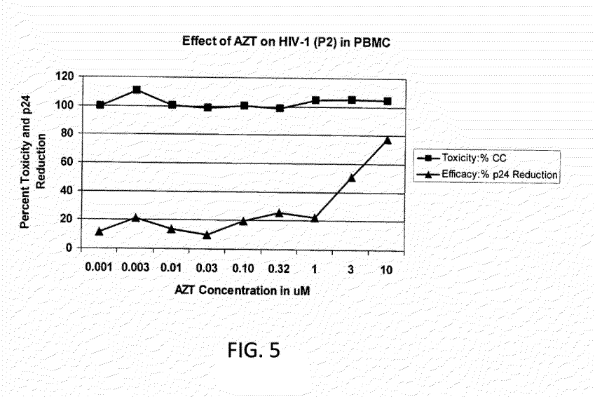

FIGS. 4-5 are graphical representations illustrating the effects of the compound of the invention and the control drug (AZT), respectively, on HIV-1 in PBMC. Tables 11-12 provide data plotted in FIGS. 4-5, respectively.

Testing in PBMCs was directed against a primary clinical isolate of HIV-1 P2, which is resistant to a variety of non-nucleoside inhibitors or HIV-1 reverse transcriptase and, in our hands, AZT. Testing AZT (control) against this virus, as expected, produced a relatively high EC₅₀ of 3 uM. Typically AZT produces EC₅₀s of less than 0.01 uM in studies of susceptible isolates in PBMCs. In this case, 90i produced an EC₅₀ of approximately 0.8 ug/ml, an IC₅₀, again, greater than 100 ug/ml and a TI of greater than 125.

TABLE 7

Testing Data For Determination of H2K-1001 (900) Suppression of P2 Replicationin PBMC

| | 100+ | 32+ | 10+ | 3+ | 1+ | 0.33+ | 0.10+ | 0.03+ | 0.01+ | VC+ |
|---|---|---|---|---|---|---|---|---|---|---|
| Mean +/-SD CV p24 Red. | 0.1530 | 0.1070 | 0.1090 | 0.4510 | 1.4266 | 1.9451 | 1.9461 | 2.1210 | 2.100 | 2.8731 |
| | 0.1820 | 0.1823 | 0.1220 | 0.3219 | 1.4567 | 1.7120 | 1.8101 | 1.9460 | 2.2130 | 2.9430 |
| | 0.2488 | 0.2177 | 0.1874 | 0.3410 | 1.1116 | 1.8460 | 1.7450 | 2.3450 | 2.2937 | 2.7600 |
| | 0.1946 | 0.1690 | 0.1397 | 0.3713 | 1.3316 | 1.8344 | 1.8337 | 2.1373 | 2.2023 | 2.8587 |
| | 0.0401 | 0.0565 | 0.0419 | 0.0697 | 0.1911 | 0.1170 | 0.1026 | 0.2000 | 0.0973 | 0.0923 |
| | 0.2525 | 0.3345 | 0.3000 | 0.1877 | 0.1435 | 0.0638 | 0.0560 | 0.0936 | 0.0442 | 0.0323 |
| | 93 | 94 | 95 | 87 | 53 | 36 | 36 | 25 | 23 | 0 |

*Drug Concentration in ug/ml.

TABLE 8

Efficacy Testing Data for AZT Suppression of P2 Replication in PBMC

| | 10+ | 3+ | 1+ | Q.32+ | 0.10+ | 0.03+ | 0.01+ | 0.003+ | 0.001+ | VC+ |
|---|---|---|---|---|---|---|---|---|---|---|
| Mean +/-SD CV % p24 Red | 0.5641 | 1.5431 | 2.2341 | 2.1786 | 2.2341 | 2.4310 | 2.5410 | 1.9152 | 2.6267 | 2.8460 |
| | 0.7416 | 1.3421 | 2.1455 | 2.3416 | 2.3519 | 2.5610 | 2.4671 | 2.8470 | 2.6849 | 2.9845 |
| | 0.6745 | 1.4541 | 2.4364 | 2.0013 | 2.4781 | 2.9461 | 2.5674 | 2.1478 | 2.4510 | 2.9541 |
| | 0.6601 | 1.4464 | 2.2720 | 2.1736 | 2.3547 | 2.6460 | 2.5252 | 2.303 | 2.5875 | 2.9282 |
| | 0.0896 | 0.1007 | 0.1491 | 0.1699 | 0.1226 | 0.2679 | 0.0520 | 0.4850 | 0.1238 | 0.0728 |
| | 0.1358 | 0.0695 | 0.0656 | 0.0782 | 0.0518 | 0.1012 | 0.0206 | 0.2106 | 0.0471 | 0.0249 |
| | 77 | 51 | 22 | 26 | 20 | 10 | 14 | 21 | 12 | 0 |

+Drug Concentration in uM.

TABLE 9

Toxicity Testing Data for H2K-1001 (90I) in PBMC Versus P2

| | 100* | 32* | 10* | 3* | 1* | 0.33* | 0.10* | 0.03* | 0.01* | CC* |
|---|---|---|---|---|---|---|---|---|---|---|
| Mean +/-SD CV % CC | 2.0185 | 1.4668 | 1.4284 | 1.3281 | 1.2837 | 1.2798 | 1.2144 | 1.6969 | 1.2592 | 1.3431 |
| | 1.3311 | 1.4605 | 1.3205 | 1.2861 | 1.3403 | 1.2969 | 1.2409 | 1.4163 | 1.3128 | 1.4220 |
| | 1.2797 | 1.4115 | 1.4232 | 1.3193 | 1.4183 | 1.3210 | 1.3410 | 1.1816 | 1.3623 | 1.3949 |
| | 1.5431 | 1.4463 | 1.3987 | 1.3102 | 1.3474 | 1.2992 | 1.2654 | 1.4316 | 1.3114 | 1.3883 |
| | 0.4125 | 0.0303 | 0.0609 | 0.0210 | 0.0676 | 0.0207 | 0.0668 | 0.2580 | 0.0516 | 0.0423 |
| | 0.2673 | 0.0299 | 0.0438 | 0.0161 | 0.0502 | 0.0159 | 0.0528 | 0.1802 | 0.0393 | 0.0305 |
| | 111 | 104 | 100 | 94 | 97 | 94 | 91 | 103 | 94 | 100 |

*Drug Concetration in ug/ml.

TABLE 10

Toxicity Testing Data for AZT in PBMC Versus P2

| | | | | | | | | | | |
|---|---|---|---|---|---|---|---|---|---|---|
| | 10+ | 3+ | 1+ | 0.32+ | 0.10+ | 0.03+ | 0.01+ | 0.003+ | 0.001+ | CC+ |
| Mean +/-SD CV % CC | 1.4841 | 1.5385 | 1.4673 | 1.2854 | 1.3539 | 1.2997 | 1.2749 | 1.3087 | 1.3486 | 1.3587 |
| | 1.3359 | 1.3532 | 1.3462 | 1.3284 | 1.3060 | 1.2844 | 1.2977 | 1.3790 | 1.3251 | 1.3885 |
| | 1.4174 | 1.3718 | 1.4308 | 1.3918 | 1.4174 | 1.4185 | 1.4911 | 1.8126 | 1.3814 | 1.3098 |
| | 1.4125 | 1.4212 | 1.4148 | 1.3352 | 1.3591 | 1.3342 | 1.3546 | 1.5001 | 1.3517 | 1.3517 |
| | 0.0742 | 0.1020 | 0.0621 | 0.0535 | 0.0559 | 0.0734 | 0.1188 | 0.2729 | 0.0283 | 0.0388 |
| | 0.0525 | 0.0718 | 0.0439 | 0.0401 | 0.0411 | 0.0550 | 0.0877 | 0.1819 | 0.0209 | 0.0287 |
| | 104 | 105 | 105 | 99 | 101 | 99 | 100 | 111 | 100 | 100 |

+Drug Concentration in uM.

TABLE 11

Effect of H2K-1001 on HIV-1 (P2) in PBMC

| H2K-1001 (ug/ml) | Toxicity % CC | Efficacy %p24 Reduction |
|---|---|---|
| 0.01 | 94 | 23 |
| 0.03 | 103 | 25 |
| 0.10 | 91 | 36 |
| 0.33 | 94 | 36 |
| 1.0 | 97 | 53 |
| 3.2 | 94 | 87 |
| 10 | 100 | 95 |
| 32 | 104 | 94 |
| 100 | 111 | 93 |

TABLE 12

Effect of AZT on HIV-1 (P2) in PBMC

| AZT (µM) | Toxicity % CC | Efficacy % p24 Reduction |
|---|---|---|
| 0.001 | 100 | 12 |
| 0.003 | 111 | 21 |
| 0.01 | 100 | 14 |
| 0.03 | 99 | 10 |
| 0.10 | 1.01 | 20 |
| 0.32 | 99 | 26 |
| 1 | 105 | 22 |
| 3 | 105 | 51 |
| 10 | 104 | 77 |

The following Table 13 summarizes the efficacy and toxicity of the antiviral agent of the invention against the HIV virus.

TABLE 13

H2K-1001 (90I) antiviral Activity in T cell lines and PBMC

| Cell Type | Virus | BC50* | IC50* |
|---|---|---|---|
| PBMC | HIV-IRF | 8.00 × 10^0 | 13.20 × 10^0 |
| CEM-SS | HIV-IRF | 2.00 × 10-2 | >1.00 × 10^2 |

*Data presented in ng/ml.

One can observe from the above that the antiviral agent of the invention, H2K-1001 (90i), yielded significant antiviral activity in two separate cell systems. It was active against a highly drug-susceptible laboratory strain of HIV-1 (RF) in an established cell line. We also evaluated the agent’s (90i’s) activity against a non-nucleoside/nucleoside resistant clinical isolate, P2 in primary peripherial blood mononuclear cells.

### Further Studies of 90I

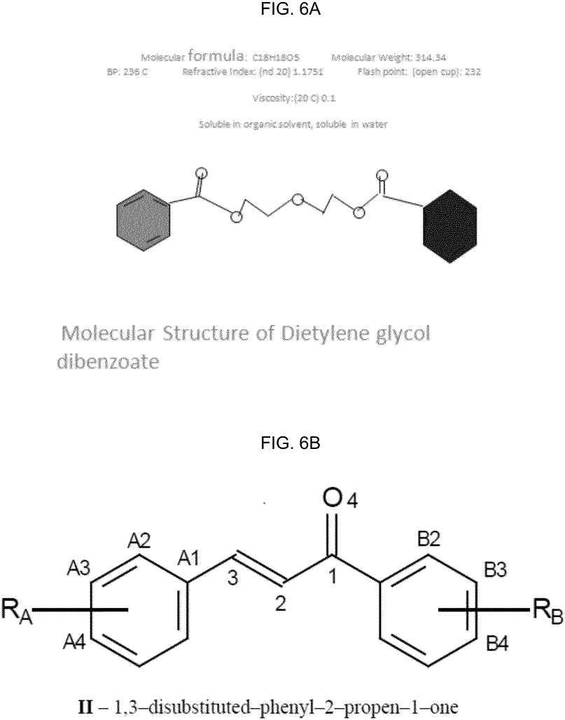

FIG. 6A illustrates a molecular structure of 90I, according to an exemplary embodiment of the present general inventive concept.

FIG. 6B illustrates a molecular structure of 90I, according to an exemplary embodiment of the present general inventive concept.

90I will be tested further. 90I may be used to treat subjects (e.g., people, animals, etc.) infected with a pathogen, which in this case is HIV. However, although treatment is the term used, treatment may also include prophylaxis (i.e. prevention), elimination, and/or vaccination, such that the treatment may inhibit replication of and/or kill the pathogen. The observed window of efficacy at a traditional herbal treatment center in Ethiopia, used in treating AIDS patients, served as the basis for undertaking the developmental investigation of the crude product for further drug development investigation.

Furthermore, although the pathogen is identified as HIV, 90I may be used to treat any pathogen including a virus, bacteria, protozoan (i.e. parasite), and/or fungal.

Bacterial pathogens may include Mycobacterium tuberculosis Tuberculosis, Bacillus anthracis Anthrax, and Staphylococcus Sepsis aureus, but is not limited thereto.

Viral pathogens may include Adenoviridae, Mastadenovirus, Infectious canine hepatitis, Arenaviridae, Arenavirus, Lymphocytic choriomeningitis, Caliciviridae, Norovirus, Norwalk virus infection, Coronaviridae, Coronavirus, Severe Acute Respiratory Syndrome, SARS-CoV, SARS-CoV-2, Torovirus, Filoviridae, Marburgvirus, Viral hemorrhagic fevers, Ebolavirus, Viral hemorrhagic fevers, Flaviviridae, Flavivirus, West Nile Encephalitis, Hepacivirus, Hepatitis C virus infection, Pestivirus, Bovine Virus Diarrhea, Classical swine fever, Hepadnaviridae, Orthohepadnavirus, Hepatitis, Herpesviridae, Simplexvirus, cold sores, genital herpes, bovine mammillitis, Varicellovirus, chickenpox, shingles, abortion in horses, encephalitis in cattle, Cytomegalovirus, infectious mononucleosis, Mardivirus, Marek’s disease, Orthomyxoviridae, Influenzavirus A, Influenza, Influenzavirus B, Influenza, Papillomaviridae, Papillomavirus, Skin warts, skin cancer, cervical cancer, Picornaviridae, Enterovirus, Polio, Rhinovirus, Common cold; Aphthovirus, Foot-and-mouth disease, Hepatovirus, Hepatitis, Poxviridae, Orthopoxvirus, Cowpox, vaccinia, smallpox, Reoviridae, Rotaviruses, Diarrhea, Orbivirus, Blue tongue disease, Retroviridae Gammaretrovirus, Feline leukemia, Deltaretrovirus, Bovine leukemia, Lentivirus, Human immunodeficiency, FIV, and SIV, Rhabdoviridae, Lyssavirus, Rabies, Ephemerovirus, Bovine ephemeral fever, Togaviridae, Alphavirus, and Eastern and Western equine encephalitis, but is not limited thereto.

Parasitic pathogens may include Plasmodium, Malaria, Leishmania, and Leishmaniasis, but is not limited thereto.

Fungal pathogens may include Aspergillis, Candida, Coccidia, Cryptococci, Geotricha, Histoplasma, Microsporidia, and Pneumocystis, but is not limited thereto.

As such, 90I may also be an anti-pathogenic compound that is applicable to different diseases and/or infections.

The Ethiopian region may be characterized by a wide range of ecological, edaphic, and climatic conditions that account for the wide diversity of its biological resources, both in terms of flora and faunal wealth. The plant genetic resources of the country exhibit an enormous diversity as seen in the fact that Ethiopia is one of the twelve Vavilov Centers of origin for domesticated crops and their wild and weedy relatives. According to recent studies, it is estimated that there are more than seven thousand species of flowering plants recorded in Ethiopia, of which at least twelve percent are probably endemic.

Medicinal plants may comprise one of the important components of Ethiopian vegetation. On record, there may be six hundred species of medicinal plants constituting a little over ten percent of Ethiopia’s vascular flora. The medicinal plants may be distributed all over the country, with greater concentration in the south and southwestern parts of the country. Woodlands of Ethiopia may be the source of most of the medicinal plants, followed by the montane grassland and/or dry montane forest complex of the plateau. Other important vegetation types for medicinal plants may be the evergreen bushland and rocky areas.

Referring to FIGS. 6A and 6B, an anti-pathogenic agent and/or the anti-pathogenic compound may be identified as 90I (or 90i). The anti-pathogenic compound may be derived from the herbal extract identified as H2K1001. H2K1001 and/or 90I may be immunogenic with unique multiple modes of action (i.e. functional or anatomical change at a cellular level, resulting from exposure of a living organism to a substance), potently effective against both reverse transcriptase and a protease (PR) enzyme (i.e. an enzyme which breaks down proteins and peptides). The essence of combination drug therapy, HAART regiments, may be its effectiveness against all HIV-1 strains that is potent enough to bring the viral load down to an undetectable level. This may be achieved by combining an RT and a PR combination synergy to affect multiple modes of action.

Referring again to FIGS. 6A and 6B, the molecular structure and molecular modeling study of H2K1001 and/or 90I may exhibit a number of properties that synergize with the outcome of the cell-based mode of action studies, early and/or late intervention. From a geometric angle, 90I may have a stable tetrameric structure like other antiretroviral compounds. From topographic 3D view, 90I has a “butterfly” moiety (i.e. a part of a molecule which is typically given a name as it can be found with other kinds of molecules as well) resembling that of the reverse transcriptase. From an angle of symmetry, 90I has a perfect vertical symmetry that divides the molecule in a mirror image isosteres (i.e. molecules or ions with similar shapes and often electronic properties) like the protease enzyme.

Additionally, 90I may be a bipolar molecule with a central domain flanked by aromatic rings (i.e. property of cylic (ring-shaped), planar structures with a ring of resonance bonds that give increased stability compared to other geometric or connective arrangements with the same set of atoms, such that the molecule has low reactivity with other substances) at both ends, as a dimer (i.e. an oligomer (a polymer whose molecules consist of relatively few repeating units) composed of two monomers that are similar in structure and jointed by a chemical bond) formation of the protease enzyme. Molecularly, 90I’s central domain may be heavily enriched with molecular oxygen and hydrogen with an extreme binding affinity (i.e. strength of the binding interaction between a single biomolecule (e.g., a protein or DNA) to its ligand/binding partner (e.g., drug or inhibitor)). The flanking aromatic rings may be in a state of dynamic pi electron (i.e. an electron which resides in the pi bond(s) of a double bond or a triple bond, or in a conjugated p orbital) resonance.

Furthermore, 90I may be a molecule with multiple charges and high affinity at various levels of chemical bonding, hydrogen bonding, covalent bonding, ionic bonding and van der wall bonding (i.e. Van der Waals force, which describes attraction and repulsions between atoms, molecules, and surfaces, as well as other intermolecular forces that differ from covalent and ionic bonding in that they are caused by correlations in the fluctuating polarizations of nearby particles).

Also, 90I may include a flexible polymerizing molecule that could easily form a dimer, a tetramer (i.e. a polymer comprising four monomer units), and a polymer that makes a multimeric web like networking. A highly active inter-penetrating polymeric nature of this molecule may provide it with unique property of rearranging the position of the binding residues, repositioning of the interacting residues, intercalating between molecules, super-imposing over a molecule, flexing in active site cleft and pockets, forming bridges between molecules, oligomerizing with the interacting molecule, causing axis rotation, out-reaching sub-units in unreachable pockets, protruding in solvent channels, and creating channels.

The fundamental underlying advantages that the 90I novel molecule has in comparison to the current treatments used for HIV may include multiple modes of action that parallels to not one, but all currently used treatments (anti-malarial, anti-HIV protease inhibitors, and interferon), highly active (HAART), proven effective against resistance, such that promoting use of this drug without the need of combinatorial drugs being required, a natural product, provides a boost to the immune system, reverse latent infection, highly effective in brain cells, non toxic, and affordable, but is not limited thereto.

As mentioned above, the initial evaluation of 90I in the CEM-SS (i.e. a human T-cell line that is highly permissive to HIV-1 infection) cell line demonstrated significant antiviral activity against the RF laboratory strain of HIV-1 (TI=IC50/EC50) of greater than 5000 suggests a highly active compound. Testing 90I in PBMC against primary clinical isolates of HIV-1 P2, which is resistant to a variety of non-nucleoside and nucleoside inhibitors of HIV-1 produced an EC50 of 0.8 ug/ml and an IC50 greater than 100 and a TI of greater than 125.

Additionally, 90I may demonstrate a highly potent antiretroviral effect against monotropic HIV-1 (macbal) in monocyte, at 50% effective concentration (EC50) of 0.03 ug/ml and a TI of greater than 4000. The AZT control had an EC50 of 0.01 uM and a TI of greater 1000. Evaluation of 90I against multi-drug resistant HIV-1 (MDR-27221) strain, a clinical isolate resistant to nucleoside reverse transcriptase inhibitors (NRTIs), non-nucleoside reverse transcriptase inhibitors (NNRTIs), and protease inhibitors (PRIs), showed a strong inhibitory activity at EC50 of 3 ug/ml and a TI of >333. Combination drug therapy with 90I and AZT as well as 90I and indinavir made synergistic antiviral effect at low concentrations 90I (0.01 ug)/AZT(0.1 uM), 90I(0.003 ug)/Indinavir (0.1 uM) of both drugs. Highly effective antiviral drug combination without antagonism at low drug concentration offers several advantages in antiretroviral therapy.

The molecular logic of a single target drug approach with a single mechanism of action (e.g., NRTI, NNRTI, INI, PRI) has become fertile ground for the evolution of resistant strains. In other words, the single target approach has not provided effective inhibition of HIV-1. However, 90I is distictly different from existing antiretroviral drugs (multiple modes and/or mechanisms of action, inhibiting HIV-1 reverse transcriptase and protease, and demonstrating early, late, and late-late modes of action), has unique properties (e.g., immune boosting), and exhibits better EC50/IC50 values than the current commercially available antiretroviral drugs with a higher therapeutic index. Additionally, unlike the other commercially available drugs, 90I is a natural product.

Since pre-historic ages, natural products like 90I have provided and continue to provide greater structural diversity than standard combinatorial chemistry. As such, natural products offer more opportunities for finding novel low molecular weight lead structures with unique mode of action, natural configuration, thermodynamic, hydrophobic, electric, stereo- geometry and topology that are active against a wide range of molecular targets.

HIV-1 may have the best complicated strategy for survival than any microbial known to man. HIV-1 may replicate more than one billion virion every day in every patient, but its replication process is error prone when the reverse transcriptase (RT) (i.e. an enzyme that catalyzes the formation of DNA from an RNA template in reverse transcription) transcribes the viral RNA to DNA.

Full blown in vitro studies may demonstrate astonishing results that surpass any known commercially available antiretroviral (ARV) and/or highly active antiretroviral therapy (HAART) drugs currently on the market.

90I may be non-toxic compound that has shown a number of antiretroviral properties that makes it a potential therapeutic agent for treatment of HIV-1 infection. In the following section, a summary of data including details of methods, tabular/graphical illustrations, and procedures have been compiled. Several studies of H2K1001 and/or 90I antiretroviral were conducted in two phases: in phase I (Ph1), H2K1001 (90I) was tested against sensitive HIV-1 (RF) and the primary clinical isolate of HIV-1 (P2). The phase II (Ph2) study evaluated H2K1001 (90I): a) against HIV-1 (multi-drug resistant) strain, b) against HIV-1 (P2) strain, c) against HIV-1 (monotropic) strain, d) two drug synergy determination, in combination with AZT and Indinavir, d) mode of action determination, both at cellular and molecular level, e) against chronically HIV-1 infected H9 cells, and f) immunogenic stimulating potential determination by the evaluation of gamma interferon production in spleenocyte of mice.

### 90I in Vitro Results

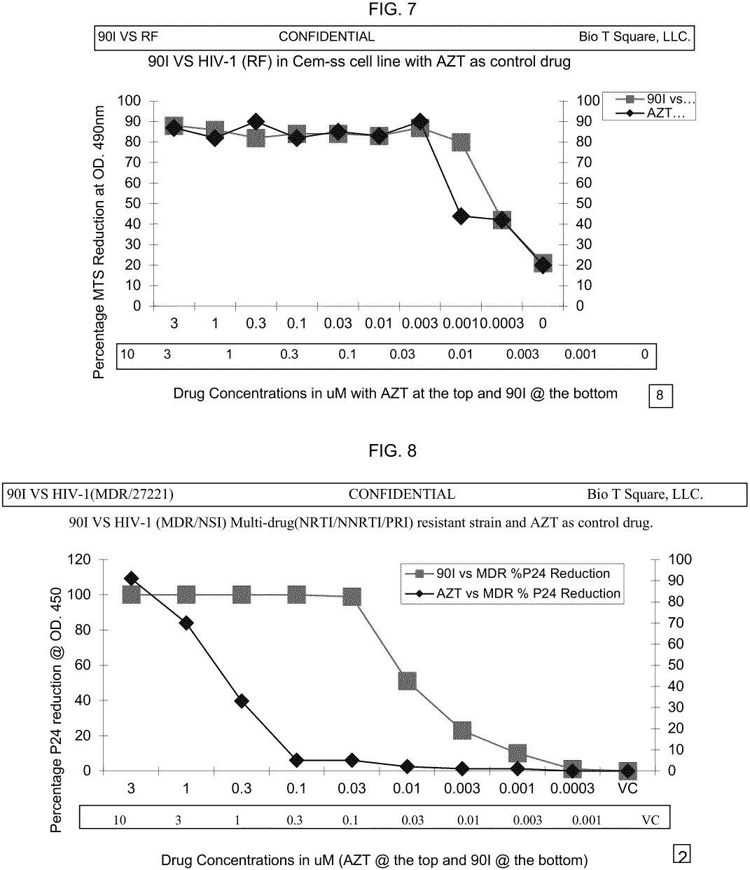

FIG. 7 illustrates a graph showing an effect of 90I compared to AZT on sensitive HIV RF strain in CEM-SS cells, according to an exemplary embodiment of the present general inventive concept.

Referring to FIG. 7, an initial evaluation of 90I and/or H2K1001 in the CEM-SS (i.e. human T-cells that are permissive to HIV-1 infection) cell line demonstrated significant antiviral activity against the RF laboratory strain of HIV-1. The purified fraction 90I may yield a 50% effective concentration (EC50) of 0.020 ug/ml. At the same time, there was no discernible drug induced toxicity in this study (50% inhibitory concentration (IC50) was greater than the high-test concentration of 100 ug/ml). The resulting therapeutic index (TI=IC50/EC50) of greater than 5000 suggests a highly active compound. An azidothymidine (AZT) (i.e. a nucleoside reverse transcriptase inhibitor or NRTI) control in this study was included to ensure the functionality of all antiviral testing, which yielded an EC50 of approximately 0.0035 uM, an IC50 greater than 10 uM and a TI of greater than 2857.

FIG. 8 illustrates a graph showing inhibitory activity of 90I compared to AZT on an MDR-27221 (MDR/NS1) HIV strain, according to an exemplary embodiment of the present general inventive concept.

Referring to FIG. 8, evaluation of 90I against the MDR HIV-1 strain (e.g., MDR-27221), a clinical isolate resistant to nucleoside reverse transcriptase inhibitors (NRTIs), non-nucleoside reverse transcriptase inhibitors (NNRTIs), and protease inhibitors (PRIs), showed a strong inhibitory activity at EC50 of 3 ug/ml and a TI of >333. The control drug, AZT, with EC50 of 3 uM failed, as expected, to inhibit the MDR strain. This finding may be significant because most of the failure of antiretroviral therapy stems from an emergence of a drug resistant virus.

Furthermore, a resistance conferring mutation has been identified for virtually all antiretroviral agents in clinical practice. A test virus parental stock originated from a genotypic antiretroviral resistance test (GART) study. Additionally, a clinical isolate was pre-confirmed for genetic and phenotypic variance prior to this test. In the previous study, 90I and/or H2K1001 may have demonstrated its effectiveness against an NRTI and/or NNRTI resistant virus, and in this study, it has significantly shown high activity against all NRTI, NNRTI, and PRI resistant varieties. Accordingly, the fact that 90I may be effective against MDR viruses with multiple modes of action, it can replace the need for combination HIV drug therapy. This finding may be highly significant because typical current single target-oriented designer antiretroviral drugs have seriously been challenged by resistant viruses. Due to natural selection, the HIV-1 virus may have developed several survival strategies that include mutation.

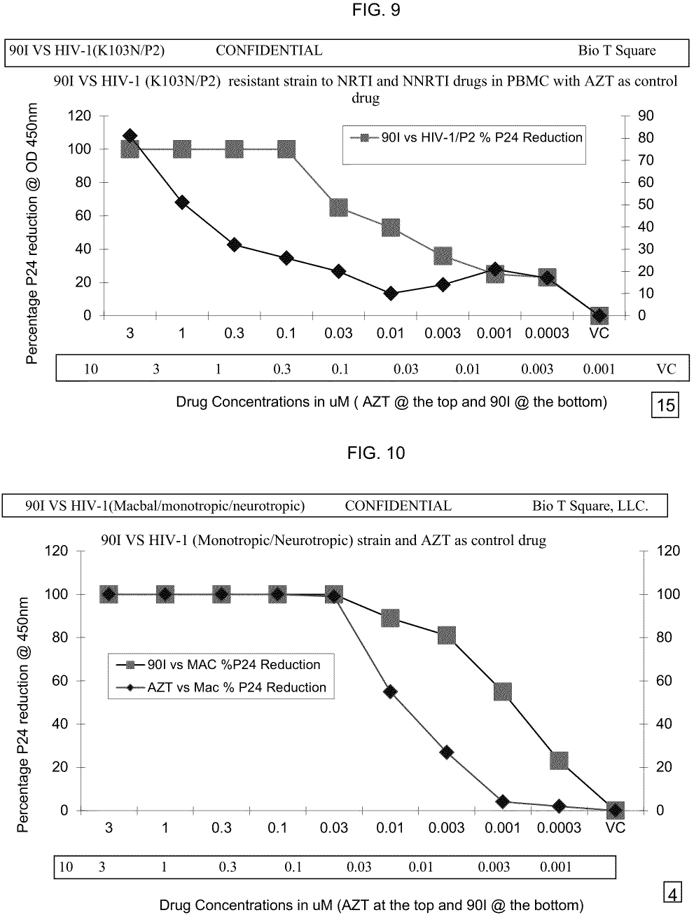

FIG. 9 illustrates a graph showing inhibitory activity of 90I compared to AZT on an K103N/P2 resistant HIV strain to NRTI and NNRTI Drugs in PBMC, according to an exemplary embodiment of the present general inventive concept.

Referring to FIG. 9, HK1001 and/or 90I was tested in PBMC against primary clinical isolates of HIV-1 P2, which is resistant to a variety of non-nucleoside inhibitors or HIV-1 reverse transcriptase. As noted previously, 90I produced an EC50 of 0.8 ug/ml, an IC50 greater than 100, and a TI of greater than 125. Also, during testing AZT produced a low efficacy at EC50 of 3 uM. Typically, AZT produces EC50s of less than 0.01 uM in studies of susceptible isolates in PBMCs.

Accordingly, the phase II (Ph2) in vitro study has clearly demonstrated and confirmed, similar to the phase I (Ph1) study, that H2K1001 and/or 90I is not only a highly active antiretroviral compound, but also immunogenic with multiple mode of actions. H2K1001 and/or 90I may include a number of properties that make it a potential therapeutic agent for treatment of HIV-1 infection.

FIG. 10 illustrates a graph showing inhibitory activity of 90I compared to AZT on an monotropic/neurotropic HIV strain, according to an exemplary embodiment of the present general inventive concept.

Referring to FIG. 10, 90I may demonstrate a highly potent antiretroviral effect against monotropic/neurotropic (i.e. a virus, toxin, or chemical that tends to attach the nervous system) HIV-1 (macbal) strains in a monocyte, at 50% effective concentration (EC50) of 0.03 ug/ml and a TI of greater than 4000. In comparison, the AZT control had an EC50 of 0.01 uM and a TI of greater than 1000.

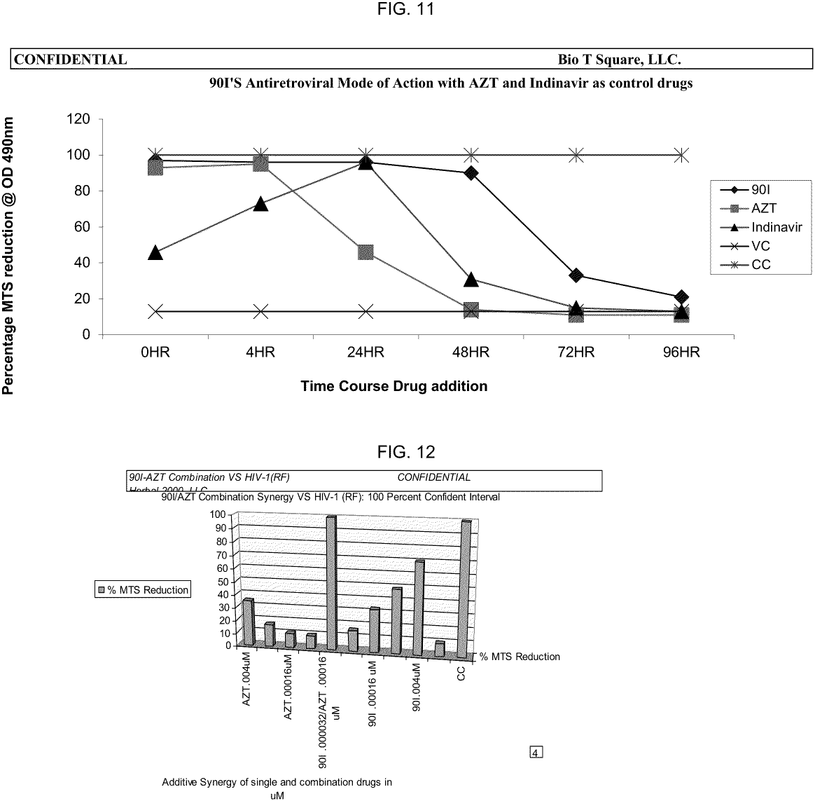

FIG. 11 illustrates a graph showing antiretroviral mode of action of 90I compared to AZT and Indinavir, according to an exemplary embodiment of the present general inventive concept.

Referring to FIG. 11, a study was performed including H2K1001 and/or 90I to demonstrate at least one mode of action thereof. Moreover, AZT and Indinavir (i.e. a protease enzyme inhibitor of HIV or PRI) were included to determine the functionality of a time course study. As the dose response curve shows, H2K1001 and/or 90I may inhibit HIV-1 both early and late. The mechanism and/or mode of action by which H2K1001 and/or 90I may inhibit HIV-1 is defined by the evaluation of a cell based time course study, and assessments of each antiretroviral study conducted for H2K1001 and/or 90I, which has been demonstrated both in phase I and phase II studies. As such, the cell based time course study is such an important tool for the determination of viral kinetics and a mode of action for a drug. The dynamics of drug-virus interaction, competitive (i.e. a drug that binds to the same site as the agonist (i.e. a drug that binds to and activate a receptor), but does not activate it) or non-competitive (i.e. a drug that binds to an allosteric site on the receptor to prevent activation of the receptor), kinetics provides a dose response curve from which a broad lead mechanism of action can be drawn. The dynamics of the interactions relate to the viral kinetic of infection and drug addition at various time points. In the various cell-based assays conducted on H2K1001 and/or 90I, it may be reasonably concluded that the compound works both early and late.

FIG. 12 illustrates a graph showing a combination of drug therapy using 90I and AZT, according to an exemplary embodiment of the present general inventive concept.

Referring to FIGS. 11 and 12, a combination drug therapy with 90I and AZT as well as 90I and Indinavir demonstrates synergistic antiviral effect at low concentrations (90I(0.01 ug)/AZT(0.1 uM), 90I(0.003 ug)/Indinavir (0.1 Um)) of both drugs. Accordingly, the drug combination is highly effective without antagonism at low drug concentration and offers several advantages in antiretroviral therapy. Also, each synergistic combination (e.g., 90I/AZT, 90I/Indinavir), lack of toxicity profile, and effectiveness of H2K1001 and/or 90I against resistant viruses, may show 90I is an excellent antiretroviral candidate for concurrent and/or alternating regimen with the current commercial drugs. Furthermore, 90I may even provide better therapeutic benefit to HIV-1 patients. A drug regimen applying a combination of drugs, such that lower or less frequent doses are used may ameliorate certain toxicities associated with each drug while retaining the clinical activity of each drug.

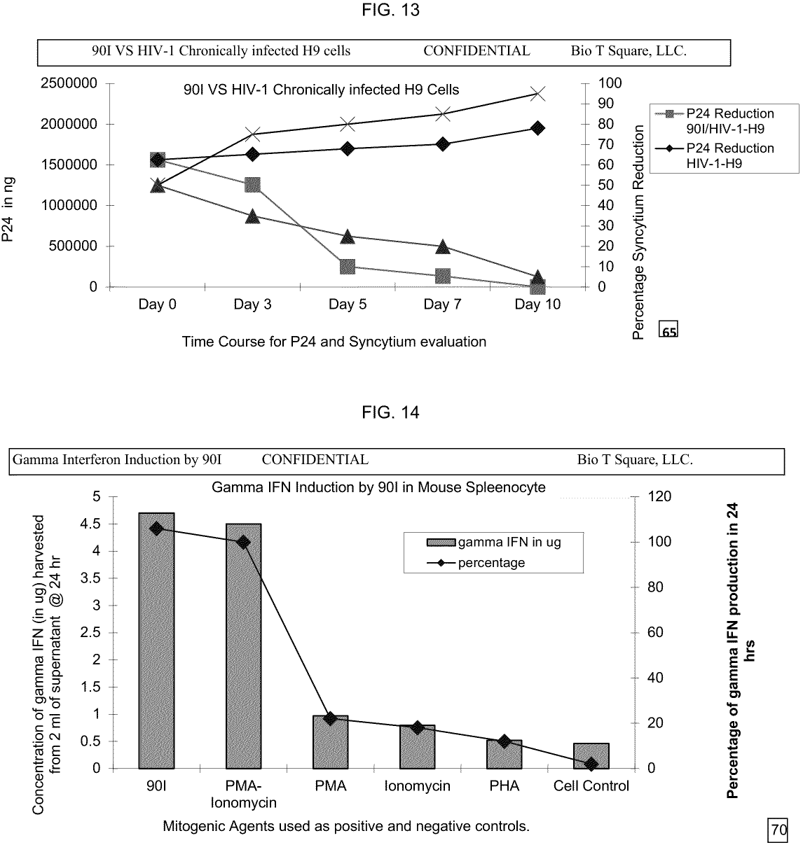

FIG. 13 illustrates a graph showing 90I effect on chronically infected H9 cells, according to an exemplary embodiment of the present general inventive concept.

Referring to FIG. 13, H2K1001 and/or 90I is compared to untreated controls, yet effectively blocked the progression of HIV-1 infection. The co-cultivation study results with H2K1001 and/or 90I demonstrating the following: (a) a reversal of viral burden from 1.7 ×10^6 pg of p24 to 5×10^2, (b) a drastic drop of syncytium formation, and (c) exponential cell growth dynamics with greater than ninety percent cell viability. These results demonstrate may highlight other significant properties of H2K1001 and/or 90I. Viral pathogenesis in co-cultivation of infected cells with uninfected target cells presents a complex system with several target sites (attachment, fusion, transcription, processing, packaging and budding).

In many documented antiretroviral studies, the co-culture system is conventionally used to isolate the virus as well as to study cell-to-cell transmission or to determine if a given compound blocks cell fusion. H2K1001 and/or 90I may use aggressive intervention that could possibly be directed to at least one of a late phase, early fusion and/or attachment, and both early and late. However, judging from the variety of 90I results, early and late mode of action is consistent to this compound. This study has shown that 90I has effectively blocked the progression of latent HIV-1 from cross transmitting infection to the uninfected cells, reversed the latent infected cells from the course of HIV-1 pathogenesis or apoptosis, and/or demonstrated early and late mode of action.

As shown above, 90I includes multiple modes of action, inhibition of early and/or late at protease, and inhibition during late-late at capsomere (i.e. a subunit of a capsid, which is the protein shell of a virus, that is an outer covering of protein that protects the genetic material of a virus) assemblage targets of HIV life cycle.

FIG. 14 illustrates a graph showing production of gamma interferon by cells receiving 90I compared to other drugs, according to an exemplary embodiment of the present general inventive concept.

Referring to FIG. 14, furthermore, 90I may stimulate the production of gamma interferon (i.e. a dimerized soluble cytokine that is the only member of the type II class of interferons and is a product of human leukocytes and human lymphocytes). More specifically, 90I may migrate from blood into tissue and differentiate into tissue macrophages, such that each of the tissue macrophages may serve as a vehicle for transporting viruses to a variety of tissues. The ability of antiretroviral agents to cross the blood brain barrier is one important consideration, since the brain acts as a sanctuary for viruses as well as a site for disease progression.

This assay may underscore 90I’s absorptions in a monocyte and/or a macrophage primary cell, such that the anti-pathogenic compound may have stability and longer pharmacokinetic half-life in ten days assay with single time point drug addition. Another significance of this result is that 90I and/or HK1001 may reinstate a dysfunctional monocyte to resume its natural functional role as a primary effector cell in the cellular immune system, effecting extensive anti-microbial and/or anti-fungal functional capability in the killing of multiple pathogens and/or other opportunistic infecting agents.

Referring again to FIG. 14, activated CD8+ cells are reported to produce high levels of gamma interferon, which may be involved in the anti-HIV-1 immune responses, contributing to both control of viral spread and concomitant lymphoid follicular lyses. An amount of gamma interferon produced by 90I was equivalent to that of the positive control, PMA-Ionomycin combination. The conclusion from this result may be that 90I stimulates cellular genes to produce gamma interferon. This finding may have a far-reaching implication and relevant in the restoration of immune system competence.

As such, 90I may be equivalent to an effective vaccine, such that 90I may modulate the immune cell signal switch from Th2 to Th1. In other words, the T-helper subset populations including the Th1 and Th2 subsets have been identified in animals and humans based on cytokine secreted. Th1 subsets favors cellular immune response by secreting cellular factors, such as IL-2, gamma interferon, and interleukin 12 (IL-12) (i.e. a cytokine that is produced by activated antigen-presenting cells, such as dendritic cells and/or macrophages). The Th-2 subset favors a humeral response, including IL-4, IL-5, IL-6 and causes activation of B cells (i.e. B lymphocytes) leading to antibody formations. Th1 provides a strong immunological response, whereas Th2 is associated with progression of HIV-1 pathogenesis. H2K1001 and/or 90I affecting gamma interferon production may be the result of Th1 subset boosting, which could have a far greater impact on reversing the course of HIV-1 infection. This study shows that 90I is not only a potent antiviral, but also an immune system booster.

These are exciting times in Ethiopia with includes many track record achievements, such as being ranked as one of the fastest growing economies for the past many years, front runner in the Global Climate Initiatives, and the top performer in achieving millennium development goals (MDG) in health sector activities, especially commended preventive measures against the main challenges of HIV, TB, and malaria. However, Ethiopia, Ethiopians, and Ethiopian herbs can advance beyond preventive measures to combat critical health problems of mankind. Additionally, HIV, TB, and malaria are the top global killers that will be cured and eliminated by the drugs discovered from at least one Ethiopian herb.

Bio-T Square is really proud to share its experience about the local research of Traditional Medicines. More specifically, the innovations of our research and extraordinary knowledge passed from Ethiopian ancestors through thousands of years and more than a thousand achievements in medicinal discovery, may justify placing Ethiopia in the history of medical research as the preeminent birth place of scientific research.

Bio-T Square appreciates the vision of the Ethiopian Government and a second growth and transformation plan (GTP) addressed to a massive industrialization economy based on scientific research and development, as well as technology and innovation. A successful short-term development and prosperity agenda for Ethiopia shall be realized through creating policy and strategy for exploiting rich biodiversity and strong collaboration between the Ethiopian Government with traditional medicine researchers and/or biotechnology firms.

Bio-T Square encourages the national initiative and commitment to build the Ethiopian Flagship Renaissance Dam through mobilizing local resources. This massive mobilization should be repeated for the development of biotechnology, which will be the biggest promise for an Ethiopian value proposition in the global economy.

The drug development scheme for 90I strictly adheres to the current FDA regulations, in vitro studies followed by toxicity animal nodel (i.e. maximum tolerable dose) will be used before human clinical trials move to phase I. Moreover, in vitro studies of 90I are complete. Animal toxicity and human clinical trials are required to get FDA certification of this drug (NID).

The objectives of this research proposal are: (1) to conduct drug formulation, safety, tolerabilty, toxicity, pharmacokinetics, and confirmation of immune modulation of 90I in animal models and (2) phase I clinical evaluation of 90I to determine safety, pharmacodynamics and pharmacokinetics of the new drug in human subjects involving Ethiopian HIV-1 patients.

### Animal Model Safety Tolerability Study of 90I:

The objective of this animal model study is to conduct drug formulation, safety, tolerability, toxicity, pharmacokinetics, pharmacodynamics, and confirmation of immune modulation of 90I in at least one animal model. This study will be conducted in Sprague-Dawley rats right before the proposed human clinical trial. The rat model is selected since it is the standard species for use in toxicology studies. The oral route was selected since this is the intended route of administration of 90I to humans. This study will be conducted in compliance with the current U.S. FDA Good Laboratory Practice (GLP) Regulations for Non-clinical Laboratory Studies (21 CFR Part 58).

A pharmaceutical composition including diethylene glycol dibenzoate, or a structurally-related derivative thereof, may be prepared, in a conventional manner. In particular, a pharmaceutical composition made in accordance with the present invention would include diethylene glycol dibenzoate, or a structural derivative thereof in an amount sufficient to provide therapeutic and/or prophylactic benefit, in combination with one or more pharmaceutically or physiologically acceptable carriers, diluents or excipients. Compositions of the present invention may be formulated for any appropriate manner for administration, including, for example, oral, nasal, intravenous or intramuscular administration. Appropriate dosages, duration and frequency of administration would be determined by known factors, such as the condition of the patient, the type and severity of the disease and the method of administration.

### REFERENCE(S)

The following reference(s), to the extent that they provide exemplary procedural or other details supplementary to those set forth herein, are specifically incorporated herein by reference.

Daids Virology Manual for HIV Laboratories, Compiled by The Division of AIDS, National Institute of Allergy and Infectious Diseases, National Institutes of Health and Collaborating Investigators, Version January 1997 (available online at http://www.niaid.nih.gov/daids/vir manual/full vir manual.pdf).

The Extended Impact of Human Immunodeficiency Virus/AIDS Research, https://doi.org/10.1093/infdis/jiy441.

THE SCIENCE OF HIV AND AIDS - OVERVIEW, https://www.avert.org/professionals/hiv-science/overview.

The present general inventive concept may include a method for treatment by boosting an immune system of a subject infected with an immunodeficiency disease, including administering to the subject in need thereof of an anti-pathogenic compound, such that the anti-pathogenic compound uses multiple modes of action against the immunodeficiency disease.

The anti-pathogenic compound may be diethylene glycol dibenzoate.

The anti-pathogenic compound may use the multiple modes of action against the immunodeficiency disease is at least one of inhibition of reverse transcriptase and protease, revitalizing at least one of a dysfunctional monocyte and a dysfunctional macrophage, modulating an immune cell signal switch from Th2 to Th1, and stimulating production of gamma interferon.

The immunodeficiency disease may be caused by HIV.

The anti-pathogenic compound may prevent at least one of early fusion, late, and both early and late stages of HIV.

The present general inventive concept may also include a method of eliminating an immunodeficiency disease from a subject, including administering to the subject in need thereof of an anti-pathogenic compound, such that the anti-pathogenic compound restores operation of an immune system.

The anti-pathogenic compound may be diethylene glycol dibenzoate.

The anti-pathogenic compound may restore operation of the immune system by inhibiting reverse transcriptase and protease.

The anti-pathogenic compound may restore operation of the immune system by boosting the immune system.

The anti-pathogenic compound may boost the immune system by revitalizing at least one of a dysfunctional monocyte and a dysfunctional macrophage.

The anti-pathogenic compound may boost the immune system by stimulating production of gamma interferon.

The anti-pathogenic compound may boost the immune system by modulating an immune cell signal switch from Th2 to Th1.

The immunodeficiency disease may be caused by HIV.

The anti-pathogenic compound may prevent at least one of early fusion, late, and both early and late stages of HIV.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

1. A method for treatment by boosting an immune system of a subject infected with an immunodeficiency disease, comprising:
administering to the subject in need thereof of an anti-pathogenic compound,
such that the anti-pathogenic compound uses multiple modes of action against the immunodeficiency disease.

2. The method of claim 1, wherein the anti-pathogenic compound is diethylene glycol dibenzoate.

3. The method of claim 1, wherein the anti-pathogenic compound using the multiple modes of action against the immunodeficiency disease is at least one of inhibition of reverse transcriptase and protease, revitalizing at least one of a dysfunctional monocyte and a dysfunctional macrophage, modulating an immune cell signal switch from Th2 to Th1, and stimulating production of gamma interferon.

4. The method of claim 1, wherein the immunodeficiency disease is caused by HIV.

5. The method of claim 4, wherein the anti-pathogenic compound prevents at least one of early fusion, late, and both early and late stages of HIV.

6. A method of eliminating an immunodeficiency disease from a subject, comprising:
administering to the subject in need thereof of an anti-pathogenic compound,
such that the anti-pathogenic compound restores operation of an immune system.

7. The method of claim 6, wherein the anti-pathogenic compound is diethylene glycol dibenzoate.

8. The method of claim 6, wherein the anti-pathogenic compound restores operation of the immune system by inhibiting reverse transcriptase and protease.

9. The method of claim 6, wherein the anti-pathogenic compound restores operation of the immune system by boosting the immune system.

10. The method of claim 9, wherein the anti-pathogenic compound boosts the immune system by revitalizing at least one of a dysfunctional monocyte and a dysfunctional macrophage.

11. The method of claim 9, wherein the anti-pathogenic compound boosts the immune system by stimulating production of gamma interferon.

12. The method of claim 9, wherein the anti-pathogenic compound boosts the immune system by modulating an immune cell signal switch from Th2 to Th1.

13. The method of claim 6, wherein the immunodeficiency disease is caused by HIV.

14. The method of claim 13, wherein the anti-pathogenic compound prevents at least one of early fusion, late, and both early and late stages of HIV.
